(19) United States
(12) Patent Application Publication
(10) Pub. No.: US 2004/0030327 A1
(43) Pub. Date: Feb. 12, 2004

(54) Surgical cauterizing instrument particularly useful as a cauterizing scalpel

(76) Inventor: Gady Golan, Hod Hasharon (IL)

Correspondence Address: Anthony Castorina, GE Ehrlich (1995) Ltd, Suite 207, 2001Jefferson Davis Highway, Arlington, VA, 22202, US

(21) Appl. No.: 10/380,267

(22) PCT Filed: Sep. 5, 2001
(86) PCT No.: PCT/IL01/00837

(51) Int. Cl. A61B 18/04

(52) U.S. Cl. 606/28

(57) ABSTRACT

A surgical instrument having a handle at one end, a metal cauterizing element, e.g., a cutting blade, at the opposite end, and an electrical heater for electrically heating the cauterizing element; the electrical heater including at least one positive temperature coefficient (PTC) heater which heats the cauterizing element and maintains a relatively constant temperature therein despite variations in the thermal load, such that the electrical power consumed by the surgical instrument automatically changes with changes in pressure applied to the cauterizing element.

### FIELD AND BACKGROUND OF THE INVENTION

The present invention relates to surgical cauterizing instruments, namely to instruments for coagulating blood in blood vessels to minimize bleeding. The invention is particularly useful in surgical scalpels including cauterizing cutting blades, and is therefore described below with respect to such an application.

Cauterizing scalpels, as widely used in surgery, include a cutting blade which is heated in order to cauterize the small blood vessels as they are cut, and thereby to minimize blood loss. The conventional cauterizing scalpels include conventional electrical heaters and manual controls to control the amount of heat generated and applied to the cutting blades. For example, if a deep incision is to be made, more heat is required to cauterize the incision than when making shallow incisions. This requires close control of the power supplied to the electrical heater for using the scalpel in an optimum manner.

### OBJECTS AND BRIEF SUMMARY OF THE INVENTION

An object of the present invention is to provide a surgical cauterizing instrument having advantages in the above respects. Another object of the invention is to provide a surgical cauterizing scalpel which may be conveniently used in an optimum manner for both cutting and cauterizing without close control of the electrical power supply.

According to one aspect of the present invention, there is provided a surgical instrument having a handle at one end, a metal cauterizing element at the opposite end, and an electrical heater for electrically heating the cauterizing element; the electrical heater including at least one positive temperature coefficient (PTC) heater which heats the cauterizing element and maintains a relatively constant temperature therein despite variations in the thermal load, such that the electrical power consumed by the surgical instrument automatically changes with changes in pressure applied to the cauterizing element.

According to another aspect of the present invention, there is provided a surgical scalpel having a handle at one end, a metal cutting blade at the opposite end, and an electrical heater for electrically heating the cutting blade; the electrical heater including at least one positive temperature coefficient (PTC) heater which heats the cutting blade and maintains a relatively constant temperature therein despite variations in the thermal load, such that the electrical power consumed by the surgical instrument automatically changes with changes in pressure applied to the cutting blade.

Positive temperature coefficient (PTC) heating elements, such as thermistors, are used in electrical heating devices, such as electrical radiators, electrical heating fans, and air conditioner heaters. They have an advantage over conventional electrical heaters in that they are self-regulating to temperature, and thus are not subject to overheating even in response to abnormal electric currents. Such PTC devices are described, for example, in U.S. Pat. Nos. 5,471,034; 5,598,502; 5,889,260; and 6,136,280, the disclosures of which are hereby incorporated by reference.

When PTC devices are used in surgical instruments in accordance with the present invention as set forth above, the temperature of the cauterizing element, or cauterizing cutting blade, is kept relatively constant so that the power consumption will automatically vary with the amount of pressure applied to the tissue since such pressure varies the thermal load applied to the PTC heater device. Thus, when the cauterizing element or cutting blade is not in contact with biological tissue, the power consumption, and therefore the heat generation, is minimum since there is a minimum thermal load. As the cauterizing element or cutting blade comes into contact with the tissue, the cauterizing element or blade is immediately thermally loaded, thus increasing the heat generation, and therefore the power consumption, in order to maintain the temperature relatively constant. The greater the pressure, or the deeper the incision, the greater will be the thermal load applied to the cauterizing element or blade, and therefore the greater will be the power supplied to the electrical heater in order to maintain the temperature constant. As soon as contact with the tissue is removed, the thermal load, and thereby the power consumption, returns to a minimum value until the cauterizing element or cutting blade is again placed into contact with a tissue to be cauterized and/or cut.

It will be appreciated that the above control in power consumption is effected automatically without the need for any external electronics, temperature sensors, or manual control elements. Such a construction thus enables the surgical instrument to be conveniently used in an optimum manner for cauterizing and/or cutting. These features also simplify the construction of the instrument, reduce its weight, and thereby facilitate its use by the surgeon.

According to further features in the preferred embodiments of the invention described below, the cauterizing element, or cutting blade, is formed with a cauterizing tip at one end exposed for cauterizing tissue, and with an electrical contact section at the opposite end disposed within the handle; and the PTC heater has an outer electrically-conductive face in direct contact with the electrical contact section of the cauterizing element.

According to further features in the described preferred embodiments, the electrical contact section of the cauterizing element or cutting blade, and the outer electrically conductive face of the PTC in contact therewith, are both flat. In the described preferred embodiments, the opposite faces of the contact section of the cauterizing element or cutting blade are both flat, and the opposite faces of the PTC heater are both electrically conductive and also flat. The contact section of the cauterizing element or cutting blade, and the opposite faces of the PTC heater, are connected to an electrical power supply by a pair of flat, spaced electrodes straddling the contact section of the cauterizing element or cutting blade, and the PTC heater.

In one described preferred embodiment, the instrument includes a single PTC heater, and in a second described embodiment, it includes two PTC heaters in contact with the opposite sides of the cauterizing element or cutting blade.

In accordance with still further features in the described preferred embodiments, the handle is integrally formed at one end of a housing, the opposite end of the housing carrying the cauterizing element or cutting blade and enclosing the PTC heater. The housing further includes an insulating bushing of heat-resistant material having an opening for receiving the cauterizing element or cutting blade. The insulating bushing is preferably of a soft, pressure-deformable material to produce a “soft feel” for the cauterizing element.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a three-dimensional view illustrating one form of surgical cauterizing instrument constructed in accordance with the present invention;

FIG. 2 is an exploded view illustrating the main components in the surgical cauterizing instrument of FIG. 1;

FIG. 3 is a longitudinal sectional view of the cauterizing instrument of FIGS. 1 and 2;

FIG. 4 is an enlarged, fragmentary, sectional view more particularly illustrating the internal structure of the cauterizing instrument of FIGS. 1-3; and

FIG. 5 is a view similar to that of FIG. 4 but illustrating a modification in the construction of the cauterizing instrument.

### DESCRIPTION OF PREFERRED EMBODIMENTS

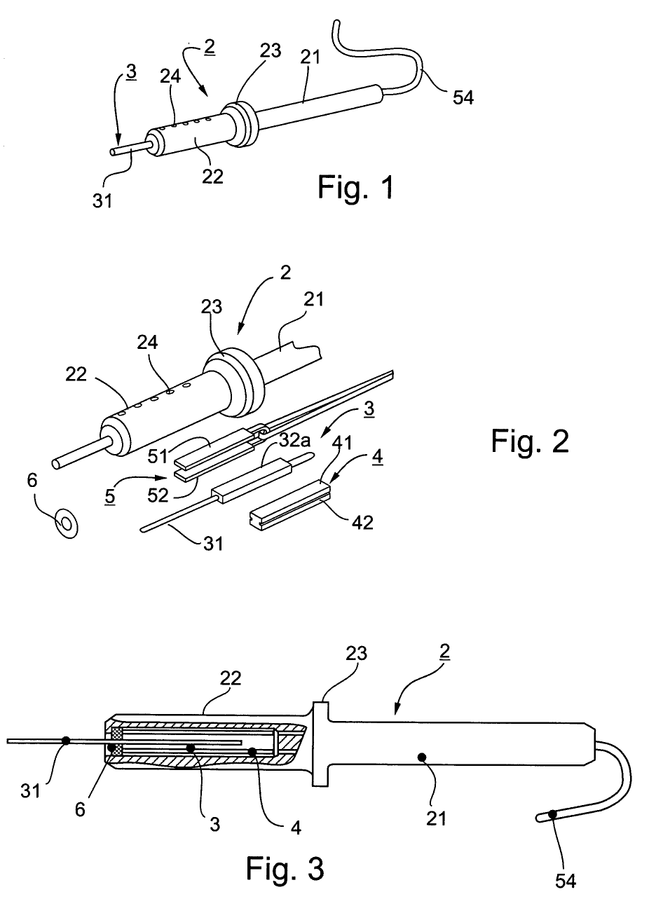

The cauterizing instrument illustrated in FIGS. 1-3 is a cauterizing surgical scalpel in which the cauterizing element is in the form of a metal cauterizing element or cutting blade to enable the instrument to be used for making incisions while cauterizing the small blood vessels as they are cut in order to minimize bleeding. As shown particularly in FIG. 2, such a cauterizing instrument includes a housing, generally designated 2; a cauterizing element in the form of a cutting blade, generally designated 3; a positive temperature coefficient (PTC) heater or thermistor, generally designated 4, for heating the cauterizing element 3; and an electrode assembly, generally designated 5, for supplying electrical current to the PTC heater 4. FIG. 2 also illustrates an insulating bushing 6 located within housing 2 and formed with an opening through which the cauterizing element 3 projects to insulate that element from the housing 2.

Housing 2 is formed of electrical and thermal insulating material. It includes a handle section 21 at one end adapted to be conveniently grasped by the surgeon, and a housing section 22 at the opposite end for carrying the cauterizing element 3 and enclosing the PTC heater 4 and the electrode assembly 5.

Housing 2 is also integrally formed with an annular rib or flange 23 between the two sections 21 and 22. Rib 23 is engageable by the fingers of the surgeon when grasping the handle 21. Rib 23 thus locates the surgeons fingers grasping handle 21 remote from the heated section 22 of the housing enclosing the heating element. Housing section 22 is preferably formed with plurality of ventilation openings 24 to vent to the atmosphere excessive heat generated within that section of the housing.

Housing 2 further includes an opening 25 at one end for the cauterizing element 3, and another opening 26 at the opposite end for the electrical cable connected to the PTC heater 4 within the cauterizing instrument.

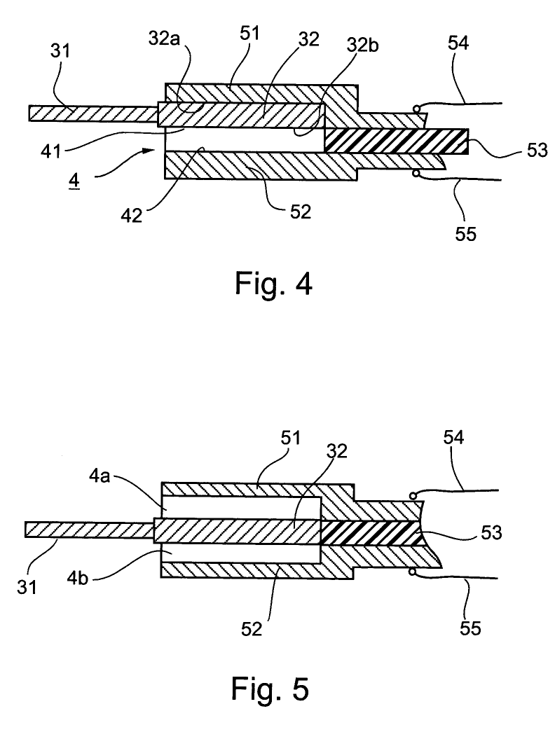

The cauterizing element 3 is in the form of a cutting blade. It has a cutting tip 31 at one end projecting through the housing 2, and an electrical contact section 32 at the opposite end within the housing 2 for making the electrical connections to the power supply via the electrode assembly 5. Electrical contact section 32 has flat opposed faces 32a, 32b (FIG. 4), to provide good electrical contact with the electrical assembly 5 and the PTC heater 4, as will be described more particularly below with respect to FIG. 4.

The PTC heater 4 is preferably one of the types described in the above-cited US Patent. Its opposite faces 41, 42 (FIG. 4) are electrically-conductive and flat, to provide good electrical and thermal contact with the electrical contact section 32 of the cauterizing blade 3, as well as with the electrode assembly 5. Preferably and electrically and thermally conductive adhesive composition, such as described in PCT/IL98/00354, published Feb. 11, 1999 as WO 99/06496, is applied to one or both faces 41, 42 of the PTC thermistor to enhance the thermal and electrical contact with the foregoing elements.

The electrode assembly 5 includes a pair of flat electrodes 51, 52 spaced from each other by an insulating spacer 53 so as to sandwich between them the electrical contact section 32 of the cauterizing cutting blade 31 and the PTC heater 4. Thus, as shown particularly in FIG. 4, the two flat electrodes 51, 52 are applied to straddle the opposite sides of the electrical contact section 32 of the cauterizing blade 31 and the PTC heater 4, with the inner flat surface of electrode 51 directly contacting the outer flat surface 32a of electrical contact section 32, and the inner contact surface of electrode 52 contacting the outer flat contacting surface 42 of the PTC heater 4. The opposite ends of the electrode 51, 52 are connected to electrical wires 54, 55 for connection to a power supply.

The insulating bushing 6 is disposed within opening 25 in the housing 2 and is formed with a central opening for passing therethrough the cutting blade 31 of the cauterizing element 3. This bushing is made of a heat-resistant insulating material sufficient to withstand the heat applied to the cutting blade 31, and to insulate that blade from the housing 24, and particularly the handle 21 grasped by the surgeon when using the cauterizing instrument. Bushing 6 is preferably made of a soft, pressure-deformable insulating material to produce a “soft feel” for cutting blade when the scalpel is used for incision and/or cauterizing purposes.

It will thus be seen that the PTC heater 4 is supplied with electrical power via the wires 54, 55, to generate heat, and transmits that heat in an efficient manner via the electrical contact section 32 to the cutting blade 31. Since the temperature is automatically kept constant in a PTC heater, the power consumption will vary with the thermal load, and therefore will be a function of the amount of pressure applied to the tissue since such pressure determines the depth of the incision and therefore the thermal load applied to the PTC heater. The illustrated cauterizing instrument thus eliminates the need for any control electronics, or temperature sensors, or manual controls to maintain the desired temperature. There is no hazard of over-heating since the PTC heater is fixed to a set temperature. The instrument provides a fast heating rate as and when required by the thermal load, and a fast cooling rate when not thermally loaded. It saves energy in terms of electrical power consumption, and is insensitive to outlet voltage variations. In addition, the illustrated instrument is of low weight and therefore can be conveniently used by the surgeon. It is also of a simplified construction which can be produced in volume and at low cost.

FIG. 5 illustrates a variation wherein the cauterizing instrument is provided with two PTC heaters, shown at 4a and 4b, on opposite sides of the electrical contact section 32 of the cauterizing blade 31. Such an arrangement applies the heat equally to the opposite faces of the electrical contact section 32 of the cauterizing blade 31 thereby more quickly and more evenly heating it in response to the required thermal load.

In all other respects, the instrument illustration in FIG. 5 involves the same construction and operation as described above, and therefore to facilitate understanding, its corresponding parts are identified the same reference numerals as used in FIGS. 1-4.

While the invention has been described with respect to two preferred embodiments, it will be appreciated that these are set forth merely for purposes of example, and that many other variations, modifications and applications of the invention may be made.

What is claimed is:

1. A surgical instrument having a handle at one end, a metal cauterizing element at the opposite end, and an electrical heater for electrically heating said cauterizing element; said electrical heater including at least one positive temperature coefficient (PTC) heater which heats said cauterizing element and maintains a relatively constant temperature therein despite variations in the thermal load, such that the electrical power consumed by the surgical instrument automatically changes with changes in pressure applied to the cauterizing element.

2. The surgical instrument according to claim 1, wherein said cauterizing element is formed with a cauterizing tip at one end exposed for cauterizing tissue, and with an electrical contact section at the opposite end disposed within said handle; said PTC heater having an outer electrically-conductive face in direct contact with said electrical contact section of the cauterizing element.

3. The surgical instrument according to claim 2, wherein said cauterizing element is a cutting blade formed with a cutting tip at said one end.

4. The surgical instrument according to claim 2, wherein said electrical contact section of the cauterizing element, and said outer electrically conductive face of the PTC heater in contact therewith, are both flat.

5. The surgical instrument according to claim 4, wherein the opposite faces of the electrical contact section of the cauterizing element are both flat, and the opposite faces of the PTC heater are both electrically conductive and also flat.

6. The surgical instrument according to claim 5, wherein said contact section of the cauterizing element, and said opposite faces of the PTC heater, are connected to an electrical power supply by a pair of flat, spaced electrodes straddling said electrical contact section of the cauterizing element and said PTC heater.

7. The surgical instrument according to claim 6, wherein said electrical contact section of the cauterizing element is directly contacted on one-face by one of said electrodes, and on its opposite face by one face of the PTC heater, the opposite face of the PTC heater being contacted by said other electrode.

8. The surgical instrument according to claim 6, wherein said faces of the PTC heater include a coating of an electrically and thermally conductive adhesive.

9. The surgical instrument according to claim 2, wherein said electrical heater includes two PTC heaters contacting opposite sides of said electrical contact section of the cauterizing element.

10. The surgical instrument according to claim 1, wherein said handle is integrally formed at one end of a housing, the opposite end of said housing carrying said cauterizing element and enclosing said PTC heater.

11. The surgical instrument according to claim 10, wherein said housing includes an insulating bushing of heat-resistant material having an opening for receiving said cauterizing element.

12. The surgical instrument according to claim 11, wherein said insulating bushing is of a soft pressure-deformable material.

13. The surgical instrument according to claim 10, wherein said housing includes an outer annular rib between said one end integrally formed with said housing, and said opposite end carrying said cauterizing element.

14. The surgical instrument according to claim 10, wherein said opposite end of the housing carrying said cauterizing element and said PTC heater is formed with ventilation openings for venting excessive heat generated within the housing.

15. A surgical instrument having a handle at one end, a metal cutting blade at the opposite end, and an electrical heater for electrically heating said cutting blade; said electrical heater including at least one positive temperature coefficient (PTC) heater which heats said cutting blade and maintains a relatively constant temperature therein despite variations in the thermal load, such that the electrical power consumed by the surgical instrument automatically changes with changes in pressure applied to the cutting blade.

16. The surgical instrument according to claim 15, wherein said cutting blade is formed with a cutting tip at one end exposed for cauterizing tissue, and with an electrical contact section at the opposite end disposed within said handle; said PTC heater having an outer electrically-conductive face in direct contact with said electrical contact section of the cutting blade.

17. The surgical instrument according to claim 15, wherein the opposite faces of the electrical contact section of the cutting blade are both flat, and the opposite faces of the PTC heater are both electrically conductive and also flat.

18. The surgical instrument according to claim 15, wherein said electrical contact section of the cutting blade, and said opposite faces of the PTC heater, are connected to an electrical power supply by a pair of flat, spaced, electrodes straddling said electrical contact section of the cutting blade and said PTC heater.

19. The surgical instrument according to claim 15, wherein said electrical heater includes two PTC heaters contacting opposite sides of said metal cutting blade.

20. The surgical instrument according to claim 15, wherein said handle is integrally formed at one end of a housing, the opposite end of said housing carrying said cutting blade and enclosing said PTC heater; said housing being further formed with an annular rib separating said handle from said opposite end of the housing.
